(19) United States
(12) Patent Application Publication
(10) Pub. No.: US 2025/0155358 A1
(43) Pub. Date: May 15, 2025

(54) MONITORING DEVICE FOR IN-SITU EVALUATION OF CORROSION UNDER INSULATION BY ELECTROCHEMICAL NOISE

(71) Applicant: Qatar Foundation for Education, Science and Community Development, Doha (QA)

(72) Inventors: Hanan Farhat, Doha (QA); Abubaker Abdelmagid, Doha (QA); Alexander Saul, Doha (QA)

(21) Appl. No.: 18/943,028

(22) Filed: Nov. 11, 2024

Related U.S. Application Data
(60) Provisional application No. 63/548,048, filed on Nov. 10, 2023.

(51) Int. Cl. G01N 17/02; G01N 27/30; G01N 33/2045

(52) U.S. Cl. CPC G01N 17/02; G01N 27/30; G01N 33/2045

(57) ABSTRACT

The disclosed technology is an electrochemical noise-based (ECN) corrosion monitoring tool (i.e., sensing device and data interpretation algorithms) for corrosion under insulation (CUI) applications. An electrochemical noise-based device for monitoring corrosion under insulation including a sensing device and a data interpretation algorithm.

### RELATED APPLICATIONS

The present disclosure claims priority to U.S. Provisional Patent Application 63/548,048 having a filing date of Nov. 10, 2023, the entirety of which is incorporated herein.

### BACKGROUND

Corrosion under insulation (CUI) is a recurrent and challenging issue for safe operations in the oil and gas industry, such as in petrochemical plants and refineries. Due to the insidious nature of the insulated systems, CUI cannot be detected easily. CUI management relies on risk-based methodologies to decide where and when to implement inspection campaigns on extensively insulated pipe networks and components. The detection rate to identifying truly active CUI sites may be improved through in-situ CUI monitoring, drastically reducing overall maintenance costs.

Electrochemical noise (EN) has been identified as a promising tool for corrosion monitoring in various environments, including those with low moisture conditions, such as atmospheric and soil corrosion. EN is sensitive to localized corrosion, which is highly likely to be associated with CUI and cannot be easily identified by other common corrosion monitoring techniques, such as electrical resistance (ER). Recently, a corrosion monitoring approach based on electrochemical current noise (ECN) coupled with machine learning was developed by the investigators and successfully applied to monitor carbon steel corrosion buried in various types of ore concentrates. Despite the low moisture contents in the ores, this method could differentiate between pitting corrosion and uniform corrosion processes.

As such, there is a need for devices for in-situ monitoring of corrosion under insulation (CUI) using electrochemical noise.

### SUMMARY

According to one non-limiting aspect of the present disclosure, an exemplary embodiment of an electrochemical noise-based device for monitoring corrosion under insulation comprising a sensing device and a data interpretation algorithm.

Additional features and advantages are described in, and will be apparent from, the following Detailed Description. The features and advantages described herein are not all-inclusive and, in particular, many additional features and advantages will be apparent to one of ordinary skill in the art in view of the description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### DETAILED DESCRIPTION

The disclosed technology is an electrochemical noise-based (ECN) corrosion monitoring tool (i.e., sensing device and data interpretation algorithms) for corrosion under insulation (CUI) applications.

In the disclosed technology, a custom-built electrochemical noise sensor comprising two identical rings made of carbon steel or stainless steel, depending on the type of pipe to be inspected for CUI. The diameter of these rings must be selected, so that the rings can fit over the tested pipe and placed under the insulation. These rings are connected with electric wires, and are used as a cathode and anode. The current measured between the rings provides information on the corrosion initiation. A potentiostat is used to collect the ECN signals. The ECN signal measurement comes in the form of current density (Acm⁻²) with time. It is used to determine the corrosion initiation. The current standard deviation is correlated with the wet/dry condition of the insulated pipes, and the severity of corrosion (localized or uniform). The current standard deviation (σI) can be used as a suitable indicator for monitoring CUI. A machine learning algorithm is developed to determine the type of corrosion (localized or uniform) using 12 variables extracted from the ECN signals, including recurrence rate, determinism, average diagonal length, length of longest diagonal line, entropy of diagonal length, laminarity, trapping time, length of longest vertical line, recurrence times of localized corrosion, recurrence times of uniform corrosion, entropy of recurrence period density and transitivity. This model allows detection and identification of the dominant CUI form.

The developed in-situ corrosion monitoring tool in the disclosed technology offers an alternative to extensive inspections. If installed at an early stage of the project, the need for manual inspections can be greatly reduced and the hitting rate can be improved.

Moreover, the key commercial applications for the disclosed technology are for any insulated pipes or devices with external insulations. Such pipes/devices can be found across industries. The disclosed technology will provide a solution to detecting corrosion in its initial stage, and to identify the type of corrosion occurring as these vary by their severity. The disclosed technology will ensure that routine inspections, where the insulation from the pipes/devices are removed and the steel surface underneath is assessed using an extremely costly and labor-demanding exercise can be reduced to occasions where corrosion has been detected by the disclosed technology.

The disclosed technology includes a measurement method that can measure and predict the extent of corrosion under insulation. The disclosed technology distinguishes between the different corrosion types, which no other method can do. Given the staggering cost of corrosion under insulation, it is expected that the disclosed technology will have significant competitive advantage, resulting in cost reduction, based on accuracy, convenient use, and high predictability. As previously noted, in the current art, corrosion under insulation (CUI) cannot be monitored to predict localized corrosion (pitting).

Moreover, the disclosed technology is highly appealing to oil and gas operators, mining operators, and petrochemical industries. The need for monitoring and prediction for maintenance schedules to prevent corrosion under insulation (CUI) has been highlighted by representatives of oil and gas, mining and petrochemical companies.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An electrochemical noise-based device for monitoring corrosion under insulation comprising:
a sensing device and
a data interpretation algorithm.

2. The electrochemical noise-based device for monitoring corrosion under insulation from claim 1, wherein the sensing device comprises two identical half-ring steel samples configured for electrochemical noise (ECN) measurement in the form of obtained electrochemical noise (ECN) signals.

3. The electrochemical noise-based device for monitoring corrosion under insulation from claim 2, wherein the sensing device is configured such that the current standard deviation (σI) of the obtained electrochemical noise (ECN) signals is correlated with the wet/dry condition within an insulating system.

4. The electrochemical noise-based device for monitoring corrosion under insulation from claim 3, wherein the data interpretation algorithm includes a mathematical model based on σI estimates corrosion rates.

5. The electrochemical noise-based device for monitoring corrosion under insulation from claim 4, wherein the data interpretation algorithm uses a machine learning model configured to identify the dominant corrosion under insulation form within the insulating system based on the obtained electrochemical noise (ECN) signals.

6. The electrochemical noise-based device for monitoring corrosion under insulation from claim 5, wherein the dominant corrosion under insulation form is one of uniform or localized.
